(19) United States
(12) Patent Application Publication
(10) Pub. No.: US 2019/0209231 A1
(43) Pub. Date: Jul. 11, 2019

(54) ABLATION BALLOON

(71) Applicant: Cook Medical Technologies LLC, Bloomington, IN (US)

(72) Inventors: Andrew P. Isch, West Lafayette, IN (US); Richard B. Sisken, P.E., West Lafayette, IN (US); Keith Milner, West Lafayette, IN (US); Joseph M. Stewart, Delphi, IN (US); Elijah Huang, Carmel, IN (US); Elizabeth St. Clair, Lexington, KY (US); Erik Nevarez, Hammong, IN (US)

(73) Assignee: Cook Medical Technologies LLC, Bloomington, IN (US)

(21) Appl. No.: 16/093,073

(22) PCT Filed: Apr. 11, 2017
(86) PCT No.: PCT/US2017/027022

§ 371 Date: Oct. 11, 2018

Related U.S. Application Data
(60) Provisional application No. 62/321,950, filed on Apr. 13, 2016.

(51) Int. Cl. A61B 18/08

(52) U.S. Cl. CPC A61B 18/082; A61B 2018/00232

(57) ABSTRACT

A tissue ablation device is provided including an inflatable balloon with a cavity. The tissue ablation device further includes a catheter with a lumen in fluid communication with the cavity of the inflatable balloon. The lumen of the catheter is configured to deliver a fluid to the cavity thereby causing the inflatable balloon to inflate and expand. The device also includes a heating element connected to the balloon, wherein the heating element is further connected to an electrical source. The heating element is configured to receive an electrical current from the electrical source. Further, the heating element is configured to raise the temperature of the inflatable balloon and the fluid when an electrical current is passed through the heating element.

### CROSS-REFERENCE TO RELATED APPLICATIONS

This application claims priority from PCT/US2017/027022, filed on Apr. 11, 2017, which claimed the benefit of the filing date under 35 U.S.C. § 119(e) of Provisional U.S. Patent Application Ser. No. 62/321,950 filed Apr. 13, 2016, the entirety of which are each incorporated by reference herein.

### FIELD

The present disclosure relates to medical devices and more specifically to endometrial tissue ablation.

### BACKGROUND

The statements in this section merely provide background information related to the present disclosure and may not constitute prior art.

Menorrhagia is an ailment characterized by excessive and/or extended menstrual bleeding. Menorrhagia can be caused by a variety of factors, such as structural abnormalities, bleeding disorders, or cancer. Various methods have been used to treat the condition such as oral contraceptives, hormone therapy, hysterectomies, drug releasing intrauterine devices, or endometrial tissue ablation. Endometrial tissue ablation is a procedure that results in the surgical destruction of the endometrial lining tissue of the uterus. Destruction of the lining tissues of the uterus may result in a significant decrease in menstrual bleeding.

A number of different methods are used to perform endometrial tissue ablation including by burning or freezing the uterine lining. For example, radio frequency energy, microwave energy, electrical energy, cryoablation, and use of a heated saline solution have all been used with varying levels of success. However, these methods have various drawbacks, including high operating costs.

Thus, it is desirable to provide an endometrial tissue ablation device and procedure that is cheap to manufacture, effective in reducing menstrual bleeding, and easy to use.

### SUMMARY

In one form of the present disclosure, a tissue ablation device is provided. The tissue ablation device comprises an inflatable balloon comprising a cavity. The device also comprises a catheter comprising a lumen in fluid communication with the cavity of the inflatable balloon, the catheter configured to deliver a fluid to the cavity thereby causing the inflatable balloon to inflate. The device further comprises a heating element connected to the balloon, wherein the heating element is further connected to an electrical source, the heating element configured to receive an electrical current from the electrical source. Additionally, the heating element is configured to raise the temperature of the inflatable balloon and the fluid when an electrical current is passed through the heating element.

Further, the tissue ablation device may have the heating element integrally attached to the balloon. The heating element may comprise printed ink that may comprise a conductive, elastomeric material. Also, the heating element may comprise an etched-foil pattern. The tissue ablation device may also further comprise a heating pad, wherein the heating element is integrally attached to the heating pad, the heating pad engageable with an outer surface of the balloon. The tissue ablation device may further comprise first and second electrical leads connected at respective first ends to the heating element, and a control box, the control box connected to the second ends of the first and second leads, the control box further connected to the electrical source, wherein the control box is configured to control the electrical current that is transferred from the electrical source through the first and second electrical leads and into the heating element. The device may also comprise a delivery sheath, wherein at least a portion of the balloon is removably disposed within a lumen of the delivery sheath.

In another embodiment, a method for ablating tissue is provided. The method comprises providing a tissue ablation device comprising an inflatable balloon comprising a cavity, a catheter comprising a lumen in fluid communication with the cavity of the inflatable balloon, and a heating element connected to the balloon. The method further comprises inserting the balloon into the patient's uterus and inflating the balloon by inserting a fluid through the catheter and into the cavity of the balloon. The method also comprises applying an electrical current through the heating element, thereby raising the temperature of the fluid and the inflatable balloon.

The method may further include the electrical current being applied through the heating element for a predetermined period of time. Further, the predetermined period of time may range from 60 seconds to 900 seconds. Also, the fluid may remain within the balloon for the predetermined period of time. The method may also include the heating element heating the balloon and the fluid to a predetermined temperature, the predetermined temperature ranging from 50 degrees Celsius to 99 degrees Celsius. The method may further comprise maintaining the balloon within the patient's uterus for a length of time after the predetermined period of time has elapsed, the length of time ranging from 12 hours to 120 hours.

### DRAWINGS

The drawings described herein are for illustration purposes only and are not intended to limit the scope of the present disclosure in any way.

FIG. 1 is a drawing of a tissue ablation balloon;

FIG. 2 is a side view of a tissue ablation balloon;

FIG. 3A is a pictorial representation of a etched-foil heating element process;

FIG. 3B is a pictorial representation of a etched-foil heating element process;

FIG. 3C is a pictorial representation of a etched-foil heating element process;

FIG. 3D is a pictorial representation of a etched-foil heating element process;

FIG. 4A is one embodiment of a tissue ablation balloon;

FIG. 4B is another embodiment of a tissue ablation balloon

FIG. 5 is a drawing of a tissue ablation device with a control box;

FIG. 6A is a representative example of a catheter for use with a tissue ablation balloon;

FIG. 6B is another representative example of a catheter for use with a tissue ablation balloon;

FIG. 7 is a representative example of a control box;

FIG. 8 is a drawing of a tissue ablation device in use with a delivery sheath;

FIG. 9 is a pictorial representation of a tissue ablation device in use;

FIG. 10 is another pictorial representation of a tissue ablation device in use;

FIG. 11 is another pictorial representation of a tissue ablation device in use;

FIG. 12 is another pictorial representation of a tissue ablation device in use;

FIG. 13 is another embodiment of a tissue ablation balloon with a heating pad;

FIG. 14 is a drawing of a heating pad for use with a tissue ablation balloon; and

FIG. 15 is a view of a tissue ablation device with a control box.

### DETAILED DESCRIPTION

The following description is merely exemplary in nature and is not intended to limit the present disclosure, application, or uses. It should be understood that throughout the drawings, corresponding reference numerals indicate like or corresponding parts and features. It should also be understood that various cross-hatching patterns used in the drawings are not intended to limit the specific materials that may be employed with the present disclosure. The cross-hatching patterns are merely exemplary of preferable materials or are used to distinguish between adjacent or mating components illustrated within the drawings for purposes of clarity.

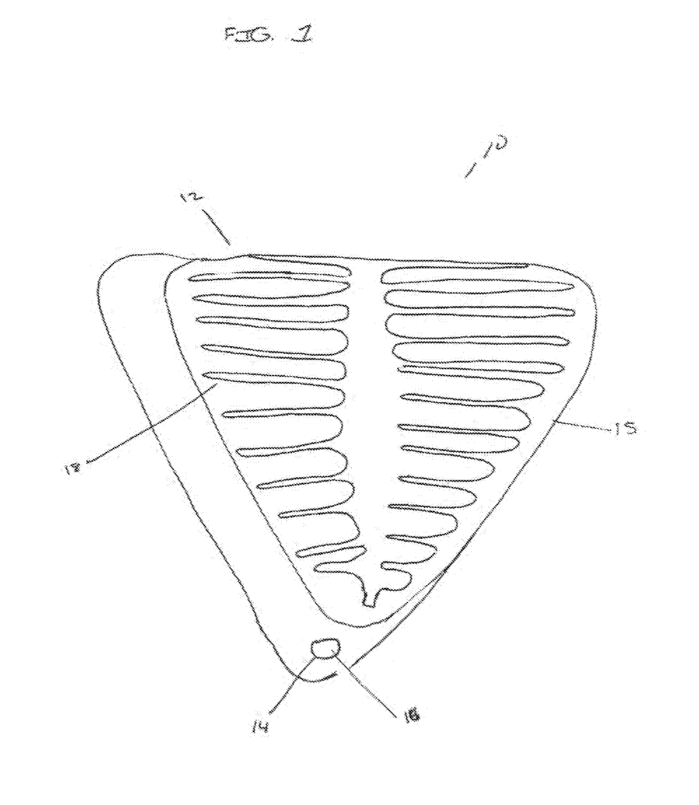

Referring to FIG, 1, a tissue ablation device 10 is provided. The tissue ablation device 10 may have a balloon 12 that may form a substantially triangular cross-section that conforms to the general shape of a uterus. The balloon 12 may have a fluid tight seal with the exception of an opening 14 that may provide fluid communication from a cavity 16 within the balloon 12 to a point external the balloon 12. The balloon 12 may be inflated with a fluid, such as saline, water, or air, through the opening 14. The balloon 12 may have a flexible surface 15 that is made of an elastic material that may stretch and expand to the shape of the uterus when the balloon 12 is inflated. Silicone may be a preferable material for the balloon 12 due to its elastic as well as thermal conductive characteristics. Other potential materials for the balloon 12 include, but are not limited to: polyurethane, polyethylene, polyamide, or other biocompatible elastomeric materials. The balloon 12 may further include a heating element 18 embedded into, etched onto, or otherwise attached to the flexible surface 15 of the balloon 12, such as with adhesive. The heating element 18 may be made of an electrically conductive material such as a biocompatible metal wire that can be attached to the surface 15 of the tissue ablation device 10. While FIG. 1 shows the heating element 18 on only one side of the balloon 12, the heating element 18 may also extend along the opposite side of the balloon 12 as well.

Further, while FIG. 1 shows the heating element 18 as a zig-zag design along the balloon 12, various other patterns may be used. Using a pattern for the heating element 18 that only covers a portion of the balloon 12 may be desirable to ensure proper expansion and contraction of the balloon 12 during inflation and delivery, even when non-elastic materials are used for the heating element 18. Further, a heating element 18 pattern that allows for even or close to even heat distribution along the entire surface of the balloon 12 (and therefore the tissue being ablated) may be preferable. Uneven heat distribution may cause some portions of the tissue to be damaged more than desired while other portions of the tissue receive insufficient heat to be properly ablated.

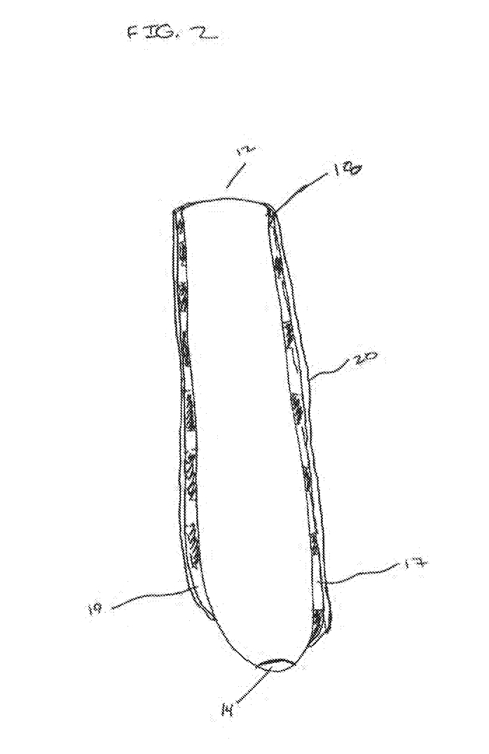

FIG. 2 shows a side view of the balloon 12 of the tissue ablation device 10. As can be seen, the flexible surface 15 of the balloon 12 may include a first surface 17 and a second surface 19. The heating element 18 may be placed on one or both of the first and second surfaces 17, 19 of the balloon 12, or along potential additional surfaces of the balloon 12, depending on the desired shape of the balloon 12. The first and second surface 17, 19 may be mated together along one or more seams, or by additional surfaces. To secure the heating element 18 to the balloon and/or to prevent direct contact of the heating element 18 with the uterine wall, a thin insulating layer 20 may be placed over the heating element 18. The insulating layer 20 may preferably be made of a thermal conductive and elastic material such as silicone, and may be casted or bonded to the balloon 12 to encapsulate the heating element 18.

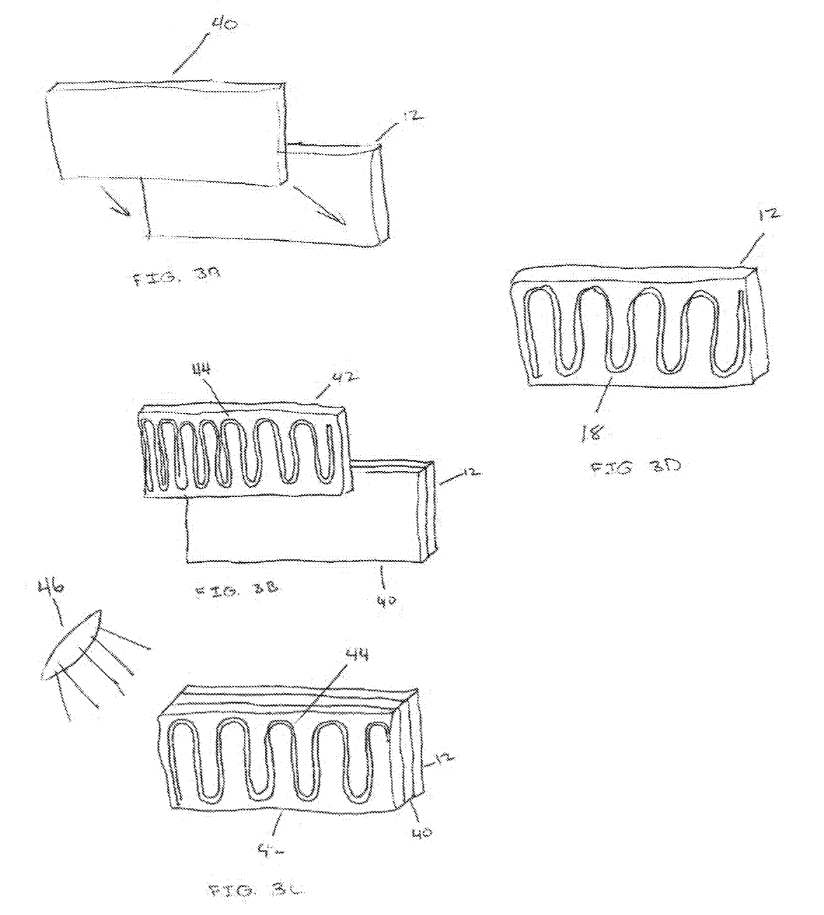

In one non-limiting example, an etched-foil design may be used for the heating element 18. In this example, the heating element 18 may be made from a metal foil 40, such as nichrome, stainless steel, or thin-film nitinol, that is patterned and etched onto the surface of the balloon 12 using photolithography. To achieve the final etched-foil design, a sheet of metal foil 40 may first be adhered to the material to be used for the balloon 12, such as silicone (FIG. 3A). Next, a photo-resistive piece of material 42 may be placed on top of the metal foil 40 (FIG. 3B). The photo-resistive material 42 may include a pattern 44 that will allow ultraviolet light to pass through a portion of the photo-resistive material 42 to the sheet of metal foil 40, while preventing ultraviolet light from passing through the non-patterned portions of the photo-resistive material 42. An ultraviolet light source 46 may then be directed towards the photo-resistive material 42 (FIG. 3C). The ultraviolet light may travel through the pattern 44 and reach portions of the foil 40 exposed by the pattern 44, thereby etching a similar pattern into the foil 40. Due to this process, the exposed portions of the foil 40 may become cured to the balloon 12, thereby making the exposed portions resistant to removal from the balloon 12 by chemical means. A series of chemical etching and stripping cycles may then be applied to the balloon 12 and metal foil 40, which results in the removal of the non-cured portions of the foil 40 from the balloon 12. What remains after this process is the balloon 12 with the heating element 18 etched directly onto the surface of the balloon 12 (FIG. 3D). While this process uses photolithography to etch the heating element 18 onto the balloon 12 or other surface, other etching processes may be used, include acid etching.

In one non-limiting example, the heating element 18 may be printed directly onto the balloon 12 of the tissue ablation device 10 using an elastomeric conductive ink. The ink may be silver or carbon based and can be applied using a printing method directly to the balloon 12. A screen printing process may be used to apply the liquid ink to the balloon 12 where it eventually dries into a solid, conductive piece of material that makes up the heating element 18. Alternatively, printing methods other than screen printing may be used to achieve the same results. The elastomeric properties of the ink may allow the heating element 18 to flex easily during insertion of the device 10 into the patient's uterus and expand easily during inflation of the balloon 12 while still maintaining strong conductive properties.

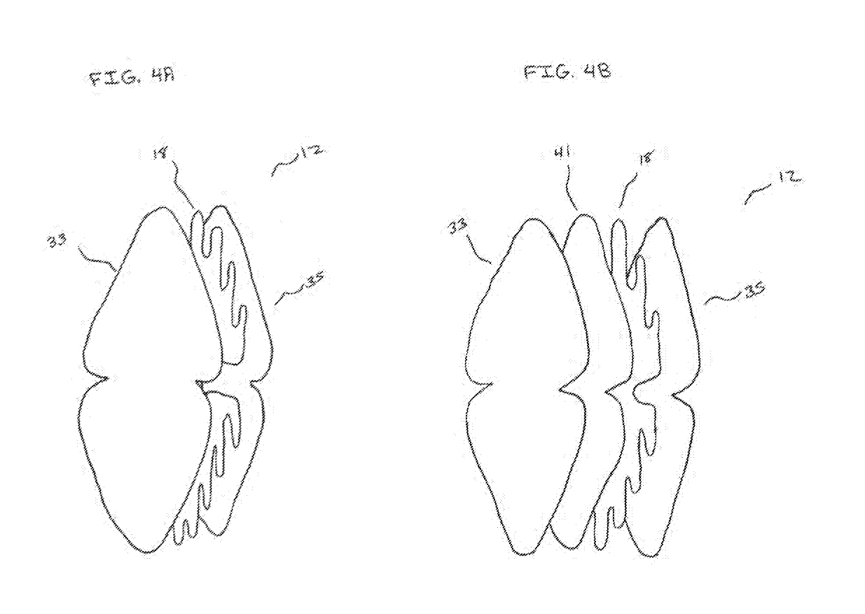

The balloon 12 may be made up of multiple layers. For example. FIG. 4A shows a balloon 12 having a heating element 18 that has been directly etched onto an inner layer 33 of the balloon 12. An outer layer 35 of the balloon 12 may be placed over the heating element 18, thereby sandwiching the heating element 18 between the inner layer 33 and outer layer 35. These layers may be made up of various materials. In one non-limiting example, the inner layer 33 may be made of polyethylene naphthalate (PEN) while the outer layer 35 may be made of silicone. FIG. 4B shows another non-limiting example, which includes an inner layer 33, an outer layer 35, a heating element 18, and an additional middle layer 41. In this example, the heating element 18 may be bonded or etched directly to the middle layer 41, while the inner and outer layers 33, 35 sandwich and protect the heating element 18 and middle layer 41. These layers may also be made up of various materials. In one non-limiting example, the inner and outer layers 33, 35 may be made of silicone while the middle layer 41 may be made of PEN. The various layers 33, 35, 41 may be bonded together in a variety of ways known in the art.

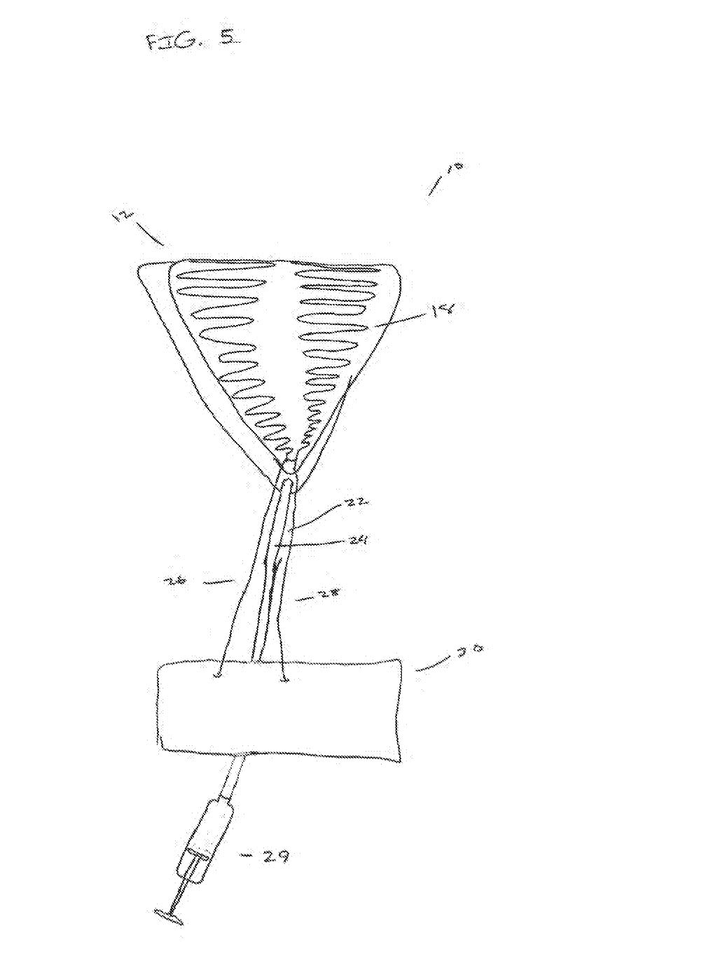

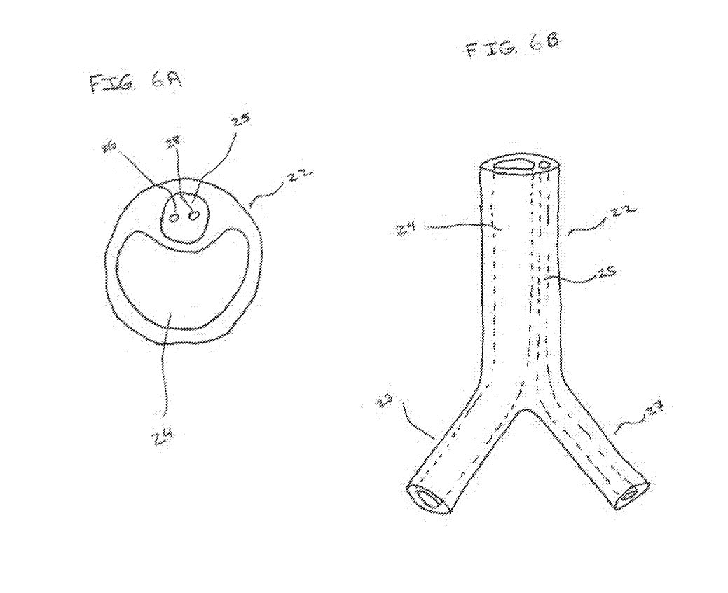

FIG. 5 shows the tissue ablation device 10 with a catheter 22 having a lumen 24 in fluid communication with the cavity 16 of the balloon 12. The catheter 22 may be placed into the opening 14 of the balloon 12 and into the cavity 16. The catheter 22 may be used to transfer fluid from a point outside the patient's body and into the cavity 16 of the balloon 12, thereby inflating the balloon 12. The catheter 22 may be connected to a fluid-filled syringe 29. The syringe 29 may be used to inject a fluid through the lumen of the catheter 22 and into the cavity 16 of the balloon 12. Other methods may or devices may be used in place of the syringe 29 to inject fluid into the balloon 12, including, but not limited to, a pump. The heating element 18 may be connected to two electrical leads 26, 28 that may be connected to a control box 30 located outside the patient's body. The control box 30 may be used to generate electrical energy that is then transferred through the electrical leads 26, 28 to the heating element 18. The heating element 18 may then convert the electrical energy into heat. The electrical leads 26, 28 may be housed within the catheter 22. For example, as shown in FIGS. 6A and 6B, the catheter 22 may include the fluid lumen 24 as well as a second lumen 25 within which the electrical leads 26, 28 are housed. At the proximal end of the catheter 22, the portion that extends outside the patient's body while in use, the catheter 22 may split off into a first branch 23 and a second branch 27. The first branch 23 may carry the fluid lumen 24 which may lead to the syringe 29 or other fluid injection device. The second branch 27 may carry the electrical leads 26, 28 which lead to the control box 30. Alternatively, the electrical leads 26, 28 may be embedded directly within the walls of the catheter 22.

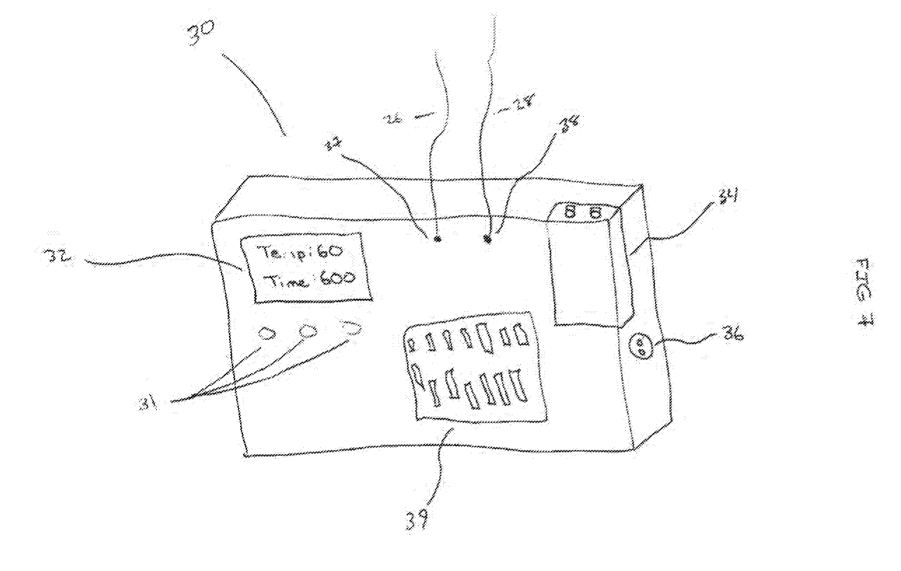

FIG. 7 shows one example of a control box 30. The control box 30 may include various controllers 31, such as buttons or knobs, used to adjust the settings of the tissue ablation device 10. An LCD screen 32 or other user interface may be used to display or output the various settings of the tissue ablation device 10 as adjusted by the controllers 31. A rechargeable battery 4 may be used to power the entire device 10. The rechargeable battery 34 may increase the portability of the tissue ablation device 10, thus making it easier to move and operate in a clinical setting. The rechargeable battery 34 may be charged by connecting a separate power source to the charge port 36, such as a wall outlet. Alternatively, disposable batteries or other sources of power may be used. The electrical leads 26, 28 may be connected to the control box 30 at two connection points 37, 38. The control box 30 may further include a circuit board 39 used to store software and control the interactions of the various features of the control box 30. The control box 30 may be used to control and vary the current, voltage, time, and other various aspects of the tissue ablation device 10. Generally, the controllers 31 may be used to input any desired changes in these variables, while the LCD screen 32 may indicate the current settings of the device 10. The control box described and shown in FIG. 7 is merely one example, and a variety of other well-known methods to control the tissue ablation device 10 may be used.

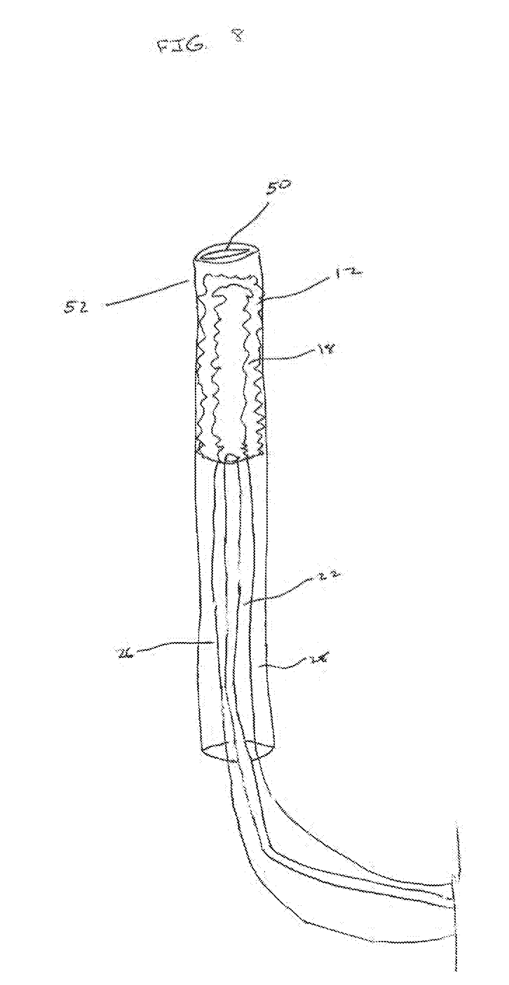

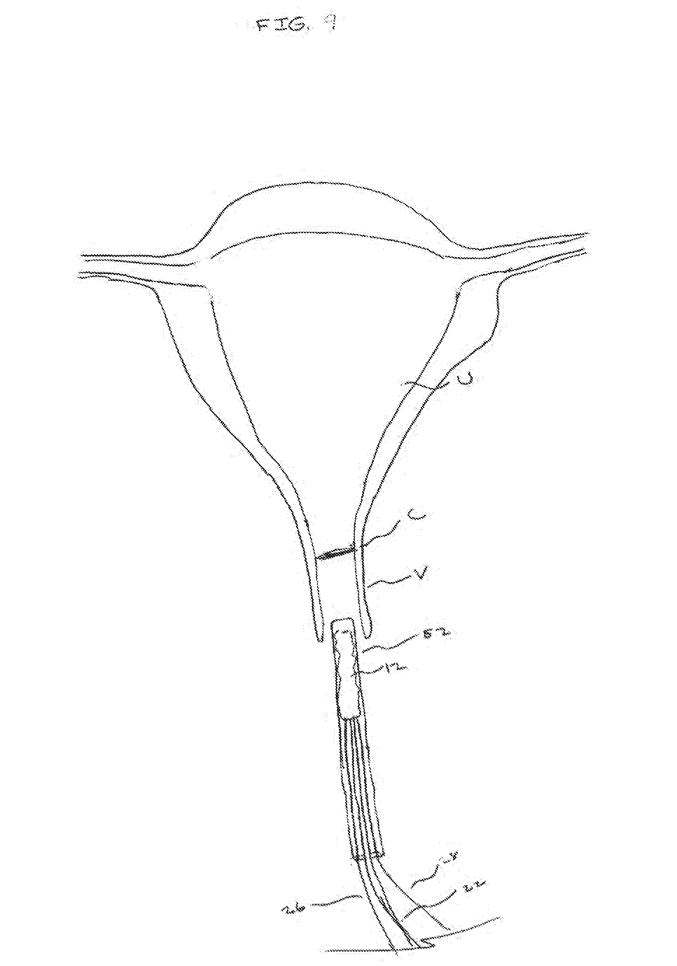

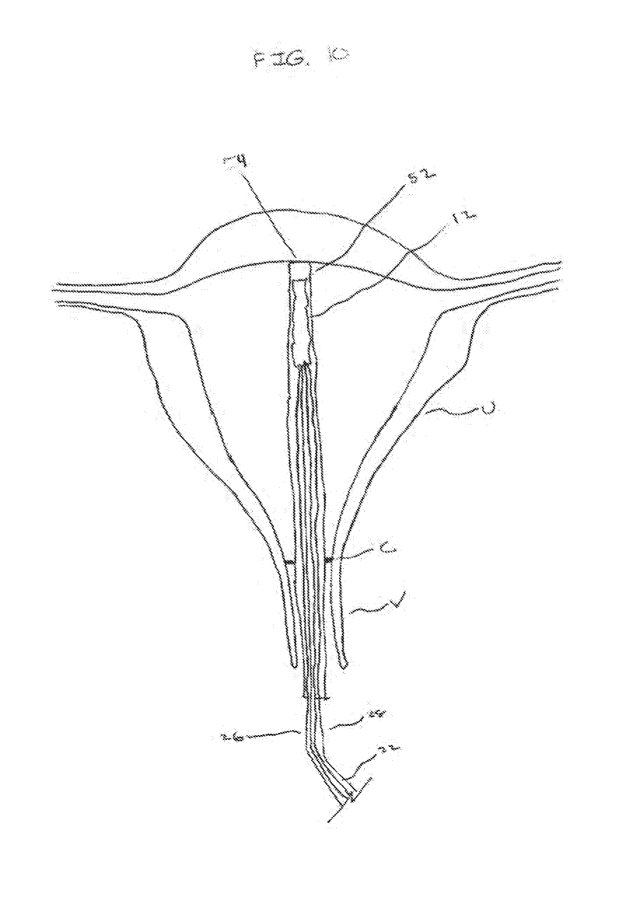

In use, the tissue ablation device 10 may have an insertion state as shown in FIG. 8. In this insertion state, the balloon 12 may be at least partially compressed into a lumen 50 of a delivery sheath 52. The catheter 22 along with the electrical leads 26, 28 may be at least partially disposed within the lumen 50 of the delivery sheath 52, while the proximal ends of the catheter 22 and electrical leads 26, 28 may be disposed outside of the delivery sheath 52. Once in the insertion state, the delivery sheath 52 along with the balloon 12 may be inserted into the patient's vagina V as shown in FIG. 9. It may be preferable for the control box 30 to be detached from the rest of the tissue ablation device 10 for the beginning of the procedure. The delivery sheath 52 may then be advanced through the cervix C. In some instances, the cervix C may need to be dilated using methods well known in the art. Once advanced past the cervix C, the delivery sheath 52 may be advanced into the patient's uterus U until the distal end 54 of the delivery sheath 52 contacts the upper wall of the uterus U as shown in FIG. 10.

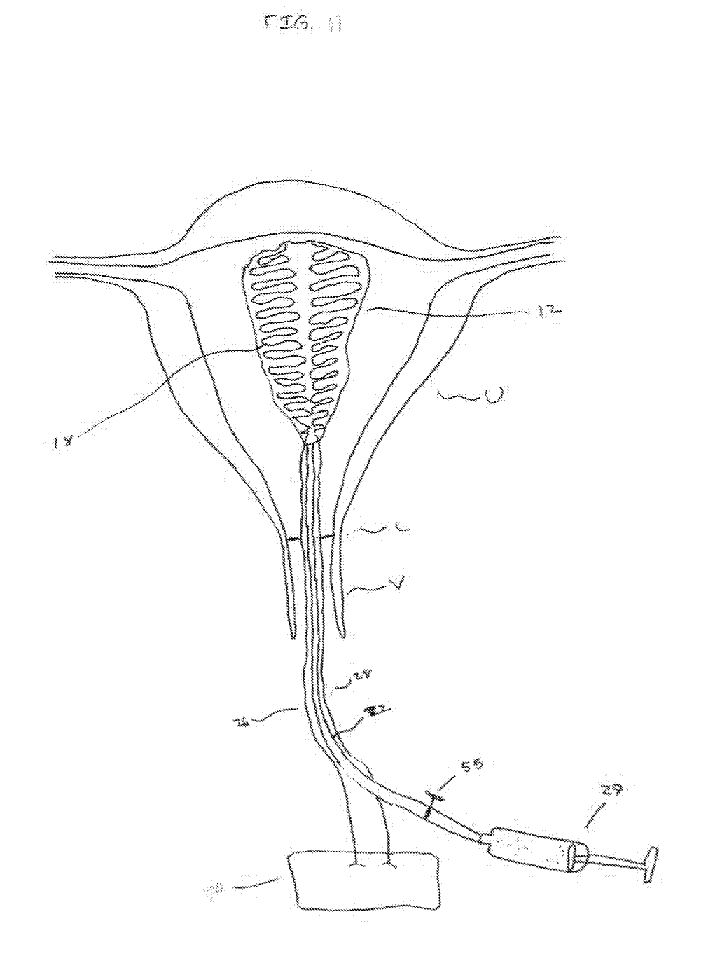

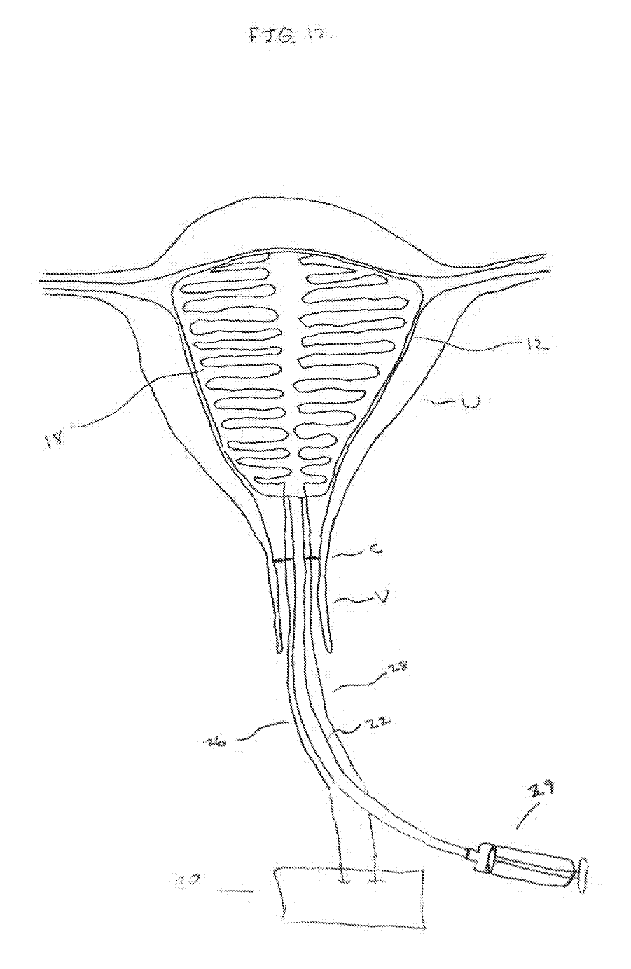

The delivery sheath 52 may then be withdrawn proximally while the balloon 12 remains stationary within the uterus U, thus causing the balloon 12 to be released from the constraints of the delivery sheath 52. As shown in FIG. 11, once the delivery sheath 52 has been completely withdrawn proximally from the patient's body, the balloon 12 is unconstrained and may now be inflated. At this point, the syringe 29 may be attached to the catheter 22. Alternatively, the syringe 29 may remain attached to the catheter for the duration of the procedure. Then, a fluid may be injected from the syringe 29 and through the catheter 22 to inflate the balloon 12. The balloon 12 may be inflated until it contacts all or most of the walls of the uterus U, as shown in FIG. 12. In one embodiment, a predetermined amount of fluid may be injected into the balloon 12 that will ensure complete inflation regardless of the size of the uterus. A stop valve 55, luer valve, or any other similar device may be used to control the flow of fluid into the catheter 22 and to maintain the balloon 12 in an inflated position once it has been properly inflated. Alternatively, if a syringe 29 is used to inject the fluid into the balloon 12, the plunger of the syringe may be lockable in a depressed position to maintain the balloon 12 in an inflated position.

If the tissue ablation device 10 was inserted into the patients body while the control box 30 was detached, the electrical leads 26, 28 may be attached to the control box 30 once the delivery sheath 52 has been proximally withdrawn from the patients body. As the balloon 12 is maintained in an inflated position, an electrical current may be applied by the control box's 30 power source 34 through the electrical leads 26, 28 and to the heating element 18. This electrical current may cause the heating element 18 to rise in temperature, which therefore may raise the temperature of the balloon 12 as well as the fluid within the balloon 12. Once the temperature of the balloon 12 reaches a certain threshold, the tissue on the walls of the uterus U may began to burn, which may result in removal of the uterine lining.

The temperature of the heating element 18 as well as the length of time the heating element 18 is activated during the procedure may be varied as desired. In one embodiment, the temperature of the heating element 18 may vary from 50 degrees Celsius to 99 degrees Celsius. Also, the activation time of the heating element 18 may vary from 60 seconds to 900 seconds. These are merely exemplary ranges, and the temperature and time may be varied further as desired. Further, the temperatures of the heating element 18 may be monitored by a thermistor (not shown) or other temperature sensing device either embedded directly into the balloon 12 or heating element 18. The thermistor may include separate electrical leads (not shown) that connect the thermistor to the control box 30, thereby allowing the user to regulate the temperature of the balloon 12 and heating element 18.

After the tissue ablation has been completed, the control box 30 may be turned off and disconnected from the electrical leads 26, 28. At this point, the stop valve 55 may be opened and the fluid may be drained from the balloon 12 to allow the balloon 12 to deflate and then be removed from the patient's body. One way to remove the tissue ablation device 10 is to reinsert the delivery sheath 52 over the catheter 22 and the balloon 12, thus reconstraining the balloon 12 into the delivery configuration. The delivery sheath 52 along with the tissue ablation device 10 may then be proximally withdrawn from the patient's body.

Alternatively, rather than immediately removing the tissue ablation device 10 after the ablation procedure has been completed, the tissue ablation device 10 may remain within the uterus while the uterine wall heals. The balloon 12 may remain inflated for a day or a period of several days to provide a protective layer over the now damaged uterine wall. The protective layer provided by the balloon 12 may help prevent or limit infection, improper or uneven healing of the uterine wall, and other potential complications during the healing process.

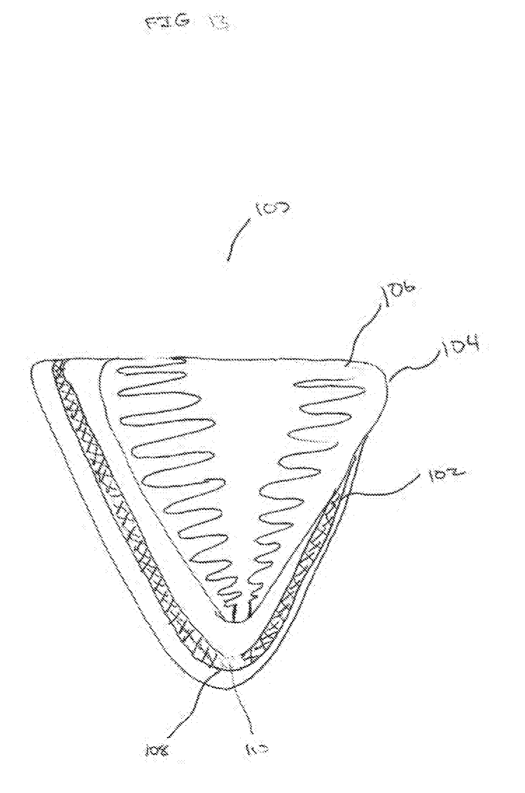

In another embodiment, as shown in FIG. 13, a tissue ablation device 100 is provided. The tissue ablation device 100 may include a balloon 102 that is disposed between two portions of a heating pad 104. The heating pad 104 may be secured to the balloon 102 using an adhesive or other similar method. The balloon 102 may include an opening 108 that provides fluid communication to a cavity 110 within the balloon 102. The balloon 102 may be made of a fluid tight, flexible, and elastic material, similar to as described in previous embodiments. The heating pad 104 may include a heating element 106 secured to the surface of the heating pad 104. The heating element 106 may be manufactured and have similar properties to the heating elements described in previous embodiments.

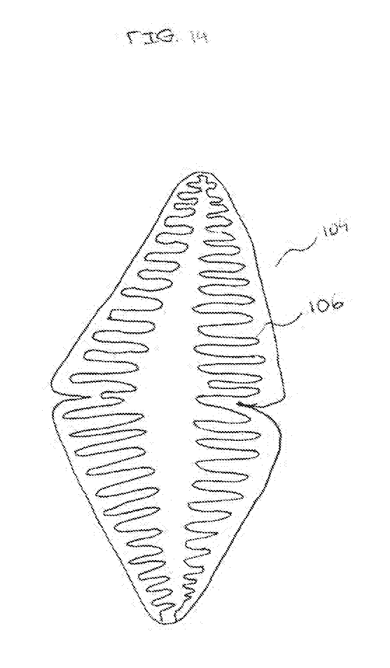

As shown in FIG. 14, the heating pad 104 may have a substantially diamond shape that is designed to fold around the balloon 102. The heating element 106 may span both, or even more, sides of the heating pad 104. The heating pad 104 may be made of a variety of biocompatible materials, but may preferably be made of an elastic and flexible material that is capable of expanding and contracting as the balloon 102 is inflated and deflated.

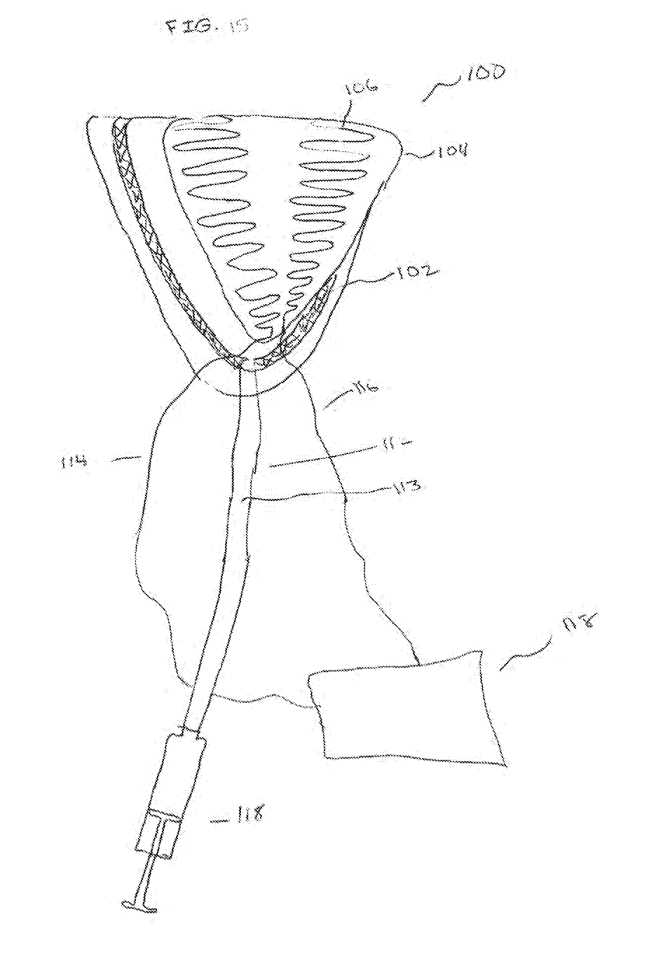

As shown in FIG. 15, the tissue ablation device 100 may further include a catheter 112 with a lumen 113. The lumen 113 may be in fluid communication with the cavity 110 of the balloon 102, thus allowing the balloon 102 to be inflated with a fluid via the catheter 112. The tissue ablation device 100 may further include two electrical leads 114, 116 connected to the heating element 106. The electrical leads may further be connected to a control box 118, which may control the heating element 106 along with the rest of the tissue ablation device 100.

The tissue ablation device 100 may be operated in substantially the same manner as described above and shown in FIGS. 8-12 with respect to the tissue ablation device 10.

While the embodiments described herein are described in reference to a tissue ablation device and process to be used to ablate the uterine lining, the embodiments are not so limited to this use. Any other ablation method, including fibroid ablation, polyablation, myometrium ablation, adenomyosis treatment, hemorrhage cauterization, and other uterine conditions, are contemplated in these embodiments.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A tissue ablation device, comprising:
an inflatable balloon comprising a flexible surface shaped to define a cavity;
a catheter comprising a lumen in fluid communication with the cavity of the inflatable balloon, the lumen of the catheter configured to deliver a fluid to the cavity thereby causing the inflatable balloon to inflate; and
a heating element connected to the balloon, wherein the heating element is connectable to an electrical source, the heating element configured to receive an electrical current from the electrical source;
wherein the heating element is configured to raise the temperature of the inflatable balloon and the fluid received within the inflatable balloon when an electrical current is passed through the heating element.

2. The tissue ablation device of claim 1, wherein:
the heating element is integrally attached to the balloon.

3. The ablation device of claim 2, wherein:
the heating element comprises printed ink.

4. The tissue ablation device of claim 3, wherein:
the printed ink comprises a conductive, elastomeric material.

5. The tissue ablation device of claim 2, wherein:
the heating element comprises an etched-foil pattern.

6. The tissue ablation device of claim 1, further comprising:
a heating pad, wherein the heating element is integrally attached to the heating pad, the heating pad engageable with an outer surface of the balloon.

7. The ablation device of claim 1, wherein:
the inflatable balloon comprises an elastic material.

8. The tissue ablation device of claim 1, further comprising:
first and second electrical leads connected at respective first ends to the heating element; and
a control box, the control box connected to the second ends of the first and second leads, the control box further connectable to the electrical source;
wherein the control box is configured to control the electrical current that is transferred from the electrical source through the first and second electrical leads and into the heating element.

9. The ablation device of claim 1, further comprising:
a protective layer disposed over the heating element.

10. The tissue ablation device of claim 1, further comprising:
a delivery sheath, wherein at least a portion of the balloon is removably disposed within a lumen of the delivery sheath.

11. The tissue ablation device of claim 1, further comprising:
a stop valve connected to the catheter, the stop valve configured to seal and unseal a portion of the lumen of the catheter.

12. The tissue ablation device of claim 1, wherein:
the flexible surface comprises first and second surfaces that are mated together along one or more seams.

13. A method for ablating tissue, comprising:
providing a tissue ablation device, comprising an inflatable balloon comprising a flexible surface shaped to define a cavity, a catheter comprising a lumen in fluid communication with the cavity of the inflatable balloon, and a heating element connected to the balloon;
inserting the balloon into a patients uterus;
inflating the balloon by inserting a fluid through the lumen of the catheter and into the cavity of the balloon; and
applying an electrical current through the heating element, thereby raising the temperature of the fluid within the cavity and the inflatable balloon.

14. The method of claim 13, wherein:
the electrical current is applied through the heating element for a predetermined period of time.

15. The method of claim 14, wherein:
the predetermined period of time ranges from 60 seconds to 900 seconds.

16. The method of claim 4, wherein:
wherein the fluid remains within the balloon for the predetermined period of time.

17. The method of claim 3, wherein:
the heating element heats the balloon and the fluid to a predetermined temperature, the predetermined temperature ranging from 50 degrees Celsius to 99 degrees Celsius.

18. The method of claim 13, wherein:
the step of providing a tissue ablation device further comprises the balloon at least partially compressed into a lumen of a delivery sheath;

19. The method of claim 18, further comprising:
releasing the balloon from a lumen of a delivery sheath by proximally moving the delivery sheath with respect to the balloon after the step of inserting the balloon into the patient's uterus.

20. The method of claim 14, further comprising:
maintaining the balloon within the patient's uterus for a length of time after the predetermined period of time has elapsed, the length of time ranging from 12 hours to 120 hours.
